(19) United States
(12) Patent Application Publication
(10) Pub. No.: US 2026/0151256 A1
(43) Pub. Date: Jun. 4, 2026

(54) WEARABLE ELECTRONIC DEVICES AND RELATED SYSTEMS AND METHODS

(71) Applicant: Somnus Technologies, Inc., Milwaukee, WI (US)

(72) Inventors: William Matthew CRONIN, Greenville, WI (US); Chloe Louise CRONIN, Greenville, WI (US); Michael Sean CRONIN, Greenville, WI (US)

(21) Appl. No.: 19/405,219

(22) Filed: Dec. 1, 2025

Related U.S. Application Data
(60) Provisional application No. 63/726,374, filed on Nov. 29, 2024.
(60) Provisional application No. 63/825,573, filed on Jun. 17, 2025.

(51) Int. Cl. A61F 5/56; A61B 5/00; A61B 5/0205

(52) U.S. Cl. CPC A61F 5/566; A61B 5/682; A61B 5/7282; A61B 5/02055; A61B 2503/10; A61B 2560/0214; A61B 2560/0462; A61B 2562/0204; A61B 2562/0219; A61B 2562/0271

(57) ABSTRACT

A wearable electronic airway device for wellness-focused and medical-focused application can integrate adjustable mandibular support for OSA management or sensor-based concussion monitoring. Embodiments can include modular sensors (e.g., PPG, accelerometers), AI analytics, and a companion app with health reports for metrics such as sleep duration, restfulness, average SpO2, resilience, and diagnostic thresholds (AHI, impacts). Embodiments thereby can assist with enhancing health outcomes through non-invasive, AI-enhanced technology.

### PRIORITY CLAIM

This application claims the benefit of U.S. Provisional Ser. No. 63/726,374 filed on Nov. 29, 2024, and U.S. Provisional Ser. No. 63/825,573 filed on Jun. 17, 2025, both entitled “WEARABLE ELECTRONIC AIRWAY DEVICE WITH ADJUSTABLE MANDIBULAR SUPPORT AND METHOD FOR MANUFACTURING,” the entireties of which are incorporated herein by reference.

### TECHNICAL FIELD

The present disclosure relates to wearable health metric technologies, specifically to oral appliances for managing obstructive sleep apnea (OSA) and snoring, as well as for monitoring concussive impacts in high-velocity sports. More particularly, the disclosure pertains to a multifunctional wearable electronic airway device with adjustable mandibular support, integrated sensors, AI-driven analytics, and methods for using, providing, managing, and manufacturing the same.

### BACKGROUND

Airway management is critical in addressing various respiratory conditions, particularly snoring and mild to moderate obstructive sleep apnea (OSA), which affects millions globally. Current devices often fail to provide adequate support for patients with a tendency for mandibular retraction, leading to airway obstruction during sleep. Existing solutions can be uncomfortable, bulky, and lack real-time monitoring capabilities. Therefore, there is a pressing need for a wearable device that effectively supports the airway while being comfortable to wear and capable of analyzing respiratory health and Smart Sensing to better deliver accurate, continuous data. Furthermore, recent consumer interest in personal health has led to a number of personal health monitoring devices being offered in the market.

The anatomy relevant to the roof of the oral cavity for custom mouthguard design according to the present disclosure includes:

Musculature
1. PALATAL MUSCLES:
   Tensor veli palatini: Tenses the soft palate, critical for maintaining patency of the airway during swallowing and breathing.
   Levator veli palatini: Elevates the soft palate, essential for isolating the nasal cavity from the oral cavity.
   Palatoglossus and Palatopharyngeus: Contribute to soft palate movements and form the arches on the lateral aspects of the oral cavity.
   Uvula Muscle: Assists in sealing the nasopharynx during swallowing and phonation.
2. OTHER MUSCLES IN PROXIMITY:
   Buccinator: Supports the lateral oral cavity wall, important for maintaining the fit of oral devices.
   Tongue Muscles (intrinsic and extrinsic): Influence the position of the tongue, which is critical in designing airway devices to avoid obstruction.

Vasculature
1. Arterial Supply:
   Greater Palatine Artery: Provides blood to the hard palate.
   Lesser Palatine Artery: Supplies the soft palate.
   Ascending Palatine Artery: Branch of the facial artery, contributes to the soft palate.
2. Venous Drainage:
   Palatine veins mirror the arteries and drain into the pterygoid venous plexus.
   Venous congestion can affect tissue dynamics relevant to airway obstruction.
3. Nerves:
   Greater and Lesser Palatine Nerves: Provide sensation to the hard and soft palates.
   Nasopalatine Nerve: Sensory innervation to the anterior hard palate.
   Glossopharyngeal and Vagus Nerves: Contribute to reflexes and sensation, ensuring airway protection.

Surface Topography of the Maxilla
1. Palatal Arch:
   The hard palate has a convex curvature with variable depth among individuals, essential for achieving a snug and functional fit.
   Landmarks include the incisive papilla and palatal rugae, which must be mapped for custom fitting.
2. Alveolar Process:
   Houses the upper dentition. Variations in the alignment and prominence of teeth influence device design.
   Overcrowding, spacing, or edentulism requires precise modeling to avoid interference.
3. Palatine Processes of the Maxilla:
   Form the anterior two-thirds of the hard palate.
   The suture line between these processes (mid-palatal suture) is crucial for device stability and alignment.
4. Maxillary Sinuses:
   Located superior to the alveolar processes. Devices must avoid unnecessary upward pressure, which could lead to discomfort or sinus irritation.

Additionally, the wearable technology market for concussive diagnosis is an emerging niche within the sports technology sector also driven by prevalence. An estimated 1.6 to 3.8 million sports-related concussions occur annually in the U.S., with high-velocity sports (e.g., football, rugby, hockey) posing significant risks. The global wearable technology market, including sports applications, is projected to grow from $157.30 billion in 2024 to $1,695.46 billion by 2032, at a CAGR of 34.6%. Increased focus on player safety, advancements in sensor miniaturization, regulatory pressures, and the need for performance optimization are driving the growth.

High-velocity sports, such as football, soccer, rugby, hockey, and motorsports, are characterized by rapid, explosive movements and significant force, posing a high risk of concussions. A concussion is a mild traumatic brain injury caused by sudden impact, collision, or rapid head movement, leading to temporary brain dysfunction. Symptoms include headache, dizziness, confusion, and nausea. Early detection and management are critical to prevent long-term neurological damage, such as chronic traumatic encephalopathy (CTE).

Current concussion management tools, such as the Glasgow Coma Scale (GCS), Sideline Concussion Assessment Tool (SCAT), and ImPACT Quick Test, rely on subjective symptom reporting, observer judgment, or post-injury assessments, increasing the risk of bias, error, or delayed diagnosis. Currently available methods and devices lack accuracy and reliability of impact data, cost and accessibility, complexity of data interpretation, and user comfort. These limitations highlight the need for objective, real-time monitoring technologies, such as wearable sensors, to enhance concussion detection and management.

### SUMMARY

The present disclosure is a wearable electronic airway device designed to provide effective airway support while preventing mandibular retraction. Equipped with adjustable features to accommodate different patient needs, the device is constructed from biocompatible materials and integrates electronic sensors for real-time analysis of biological signals. The manufacturing method ensures ease of production while maintaining quality and safety standards. This innovative solution aims to improve patient outcomes in managing airway-related conditions.

The wearable electronic airway device of the present disclosure includes an adjustable mandibular support and represents a significant advancement in the field of airway management. By integrating biomechanical principles with innovative electronic features, this device addresses the critical need for effective support in preventing mandibular retraction, a common issue in individuals experiencing respiratory challenges. The device can be fabricated from a biocompatible material (such as a polyethylene or any medical grade high-density material) for comfort and safety. In the maxilla, the sensor(s) can be positioned on the palatal gingiva adjacent to the canine or premolar teeth, a site selected for its thick, highly vascularized mucosa and proximity to the greater palatine artery, which can provide robust perfusion and accurate SpO2 readings. A corresponding optimal location in the mandible can be the lingual gingiva immediately adjacent to the lower canine and first premolar teeth, a site selected for its moderately thick, highly vascularized mucosal, rich sublingual arterial plexus and mental artery. Placement immediately adjacent to the teeth can minimize tongue interference and unconscious displacement while maximizing wearer comfort. When comfort, perfusion, and signal quality are jointly considered, the canine palatal region can be an advantageous intraoral location for pulse oximetry. Optional upper and lower shelves proximal to the teeth can further improve retention and enable multimodal biological signal analysis.

The methods of manufacturing outlined in this application demonstrate a clear and effective process for creating a reliable product while safeguarding the integrity of the electronic components. Through careful engineering and quality assurance, embodiments of this disclosure are designed to meet the demands of both personal users and clinicians in clinical settings effectively.

The unique combination of airway support, sensor integration, and user adaptability not only presents a novel solution to a pressing problem but also opens avenues for further research and development in wearable health technology. Continued exploration of its capabilities could lead to enhancements in patient outcomes and broader applications in health monitoring and management.

Additionally, devices as disclosed herein are uniquely positioned to address challenges related to concussion detection and management by offering a multifunctional, AI-driven platform that integrates seamlessly into both clinical and sports environments.

The detailed technology and example embodiments implemented for the subject disclosure are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present disclosure.

### BRIEF DESCRIPTION OF THE DRAWINGS

Embodiments of the present disclosure may be better appreciated upon considering the drawings, of which:

FIG. 1A depicts an embodiment of the WEAD of the present disclosure.

FIG. 1B depicts another embodiment of the WEAD of the present disclosure.

FIG. 1C depicts another embodiment of the WEAD of the present disclosure.

FIG. 1D-1 depicts another embodiment of the WEAD of the present disclosure.

FIG. 1D-2 depicts another embodiment of the WEAD of the present disclosure.

FIG. 1E depicts another embodiment of the WEAD of the present disclosure.

FIG. 1F depicts another embodiment of the WEAD of the present disclosure.

FIG. 1G depicts another embodiment of the WEAD of the present disclosure.

FIG. 1H depicts another embodiment of the WEAD of the present disclosure.

FIG. 2 depicts another embodiment of the WEAD of the present disclosure.

FIG. 3 depicts another embodiment of the WEAD of the present disclosure.

FIG. 4 depicts another embodiment of the WEAD of the present disclosure.

FIG. 5 depicts another embodiment of the WEAD of the present disclosure.

FIG. 6 depicts an embodiment of a molding stand and charger with a WEAD of the present disclosure.

FIG. 7 depicts another embodiment of a WEAD of the the present disclosure.

FIG. 8A depicts a block diagram of a system of the present disclosure.

FIG. 8B depicts a block diagram of a system of the present disclosure.

FIG. 9 depicts another block diagram of a system of the present disclosure.

FIG. 10 depicts a flowchart of data collection and processing of the present disclosure.

FIG. 11 depicts a manufacturing process of the present disclosure.

While the embodiments of this disclosure are amenable to various modifications and alternative forms, specifics thereof have been shown by way of example in the drawings and will be described in detail. It should be understood, however, that the intention is not to limit the disclosure to the particular example embodiments described. On the contrary, the disclosure covers all modifications, equivalents, and alternatives falling within the spirit and scope of the claims.

### DETAILED DESCRIPTION

As the inventor of this innovative wearable electronic airway device (WEAD), I have identified critical unmet needs in both sleep health and sports safety. The wearable technology market for measuring health metrics is rapidly growing. The Smart Oral Appliances as disclosed and described herein include wearable health metric technologies (HMT) designed for at least two distinct applications: (1) the management of snoring and obstructive sleep apnea (OSA) through mandibular advancement and comprehensive sleep metric monitoring, and (2) the accurate and precise measurement of impact forces and physiological responses for concussion monitoring in high-velocity sports. The devices described and disclosed herein integrate advanced sensor technology, artificial intelligence (AI) algorithms, Bluetooth connectivity for real-time feedback, and wireless recharging, offering desirable battery life (e.g., up to 7 days or 168 hours or more) or more in some embodiments Devices according to embodiments of the present disclosure offer a multifunctional, non-invasive solution that enhances patient outcomes and athlete safety.

The wearable technology market for measuring health metrics is rapidly expanding, driven by increasing awareness of conditions like obstructive sleep apnea (OSA) and the risks of concussive injuries in high-velocity sports. Traditional solutions for OSA, such as continuous positive airway pressure (CPAP) machines, are effective but suffer from low compliance due to discomfort, noise, and portability issues. Mandibular advancement devices (MADs) offer a non-invasive alternative but lack real-time monitoring, precision adjustment, and integration with advanced analytics. Similarly, in sports, concussion detection relies on subjective assessments or basic helmet sensors, which fail to capture intraoral impact data or physiological responses accurately. Existing mouthguards are often rigid, non-customizable, and lack upgradability for new sensor technologies.

Embodiments of this disclosure can address these gaps, while also addressing other unmet needs in the industry, by providing a platform with embodiments for: (1) wellness-focused monitoring of sleep and activity metrics, marketed over-the-counter (OTC) to promote general health without diagnostic claims, and (2) medical-focused monitoring for OSA management or concussion diagnostics, requiring regulatory clearance.

Embodiments of the WEAD of this disclosure integrate one or more research-grade sensors, proprietary AI algorithms for predictive analytics, Bluetooth or other wireless connectivity for data sharing, and wireless recharging with desirable battery life.

Embodiments of the WEAD of this disclosure can be water-resistant to IP67 standards, ensuring durability in humid oral environments and high-sweat or water-related sports activities, with sealed sensor housings that withstand submersion up to 1 meter for 30 minutes in one example embodiment.

Embodiments of this disclosure therefore can provide numerous advantages with respect to conventional approaches, including enhanced patient outcomes, improved athlete safety, and various positive lifestyle modifications through an AI-enhanced personalized health optimization platform that delivers tailored insights for both clinical diagnostics and everyday wellness guidance.

As previously mentioned, there are significant unmet needs in the market. A comprehensive review of market data as of October 2025 included the following:
OSA Prevalence: Approximately 83.7 million adults in the U.S. (based on a 2025 Sleep Medicine Reviews study adjusting for obesity and underdiagnosis) and ˜1 billion adults worldwide (aligned with 2023 Lancet Respiratory Medicine estimates, adjusted for population growth) suffer from mild to severe OSA, with snoring affecting up to 94% of OSA patients.
Wearable Medical Devices Market: The global market was valued at $45 billion in 2024, projected to reach $151.8 billion by 2029 at a 27.5% CAGR, per Grand View Research 2025, driven by demand for at-home monitoring and chronic disease management.
Sleep Tracking Devices Market: Valued at $26.7 billion in 2023 with an 11.7% CAGR, per MarketsandMarkets 2025, reflecting consumer interest in wellness and sleep optimization.
Sports Concussions: 1.6 to 3.8 million sports-related concussions occur annually in the U.S., per CDC and Brain Injury Research Institute 2025 estimates.
Wearable Technology Market: Projected to grow from $84.2 billion in 2024 to $186.14 billion by 2030 at a 13.6% CAGR, per Statista 2025, fueled by safety regulations and sensor advancements. These figures align with industry reports, correcting earlier overestimations (e.g., sports wearable market size) for accuracy and credibility.

Conventional approaches, such as basic mandibular advancement devices (MADs) and impact-sensing mouthguards, lack one or more significant features of embodiments disclosed herein, such as the integration of AI, modular sensors, and dual-use functionality. Furthermore, current approaches consider sleep and sports safety to be distinct fields, such that no existing approach attempts to address the needs of both applications. In other words, the synergy of intraoral positioning for dual metrics (respiratory and impact) is unexpected.

Current treatments, such as continuous positive airway pressure (CPAP) machines, are the gold standard for moderate to severe OSA but face significant limitations, including physical discomfort, compliance challenges, technical issues, and high costs. Traditional oral appliances, such as mandibular advancement devices (MADs), offer a non-invasive alternative but lack advanced monitoring capabilities, accuracy validation, and regulatory approval.

Accordingly, embodiments of the device disclosed herein address these limitations by integrating one or more precision sensors, AI analytics, and user-friendly design to provide a personalized, accurate, and comfortable solution for snoring and mild to moderate OSA.

The wearable technology market for concussive diagnosis is an emerging niche within the sports technology sector also driven by prevalence. An estimated 1.6 to 3.8 million sports-related concussions occur annually in the U.S., per CDC and Brain Injury Research Institute 2025 estimates. The global wearable technology market, including sports applications, is projected to grow from $84.2 billion in 2024 to $1,695.46 billion by 2032, at a CAGR of 13.6% per Statista 2025, fueled by safety regulations and sensor advancements.

High-velocity sports, such as football, rugby, soccer, hockey, and motorsports, are characterized by rapid, explosive movements and significant force, posing a high risk of concussions. A concussion is a mild traumatic brain injury caused by sudden impact, collision, or rapid head movement, leading to temporary brain dysfunction. Symptoms include headache, dizziness, confusion, and nausea. Early detection and management are critical to prevent long-term neurological damage, such as chronic traumatic encephalopathy (CTE).

Current concussion management tools, such as the Glasgow Coma Scale (GCS), Sideline Concussion Assessment Tool (SCAT), and ImPACT Quick Test, rely on subjective symptom reporting, observer judgment, or post-injury assessments, increasing the risk of bias, error, or delayed diagnosis. Currently available methods and devices lack accuracy and reliability of impact data, cost and accessibility, complexity of data interpretation, and user comfort. These limitations highlight the need for objective, real-time monitoring technologies, such as wearable sensors, to enhance concussion detection and management. Devices as disclosed herein are uniquely positioned to address these challenges by offering a multifunctional, AI-driven platform that integrates seamlessly into both clinical and sports environments.

Existing mouthguards and oral appliances often suffer from limited customizability and upgradability. For instance, sensors are typically fixed, preventing easy replacement or adaptation to new technologies. Boil-and-bite mouthguards provide accessible fitting but rarely integrate advanced, interchangeable sensors. One-piece designs for upper-jaw use in high-velocity sports lack mandibular control but could benefit from modular sensors and adjustments for enhanced fit and functionality.

The present disclosure provides a wearable electronic airway device, referred to herein as a WEAD. Though referred to herein as a “device,” the WEAD can be considered to be system in that in can comprise a wearable device with wireless communications capabilities to an external computing device (e.g., other wearable device, smart phone, tablet, computer), and which further can include cloud-enabled or resident algorithms, data, or software. Thus, the use of either “device” or “system” herein is not limiting with respect to any overall configuration consisting of or comprising the WEAD.

Embodiments of the WEAD can be provided in multiple configurations, which can include: a wellness-focused configuration for non-diagnostic lifestyle monitoring, and a medical or clinical configuration for diagnosing and managing OSA or concussions. Each configuration can include an upper tray, a lower tray (in OSA-focused embodiments), a dual adjustment mechanism (such as one or more of a titration screw, ratchet, spring, or other compatible mechanism for, e.g., 0.1 mm increments in OSA versions), modular sensors (e.g., one or more of a photoplethysmogram (PPG) sensor, an accelerometer, a gyroscope, a temperature sensor, a snore sensor such as a piezo sensor or microphone), a battery (rechargeable in some embodiments and can be replaceable in other embodiments), and HIPAA-compliant wireless connectivity (e.g., Bluetooth, Zigbee, near-field communication (NFC), Wi-Fi, among others). Embodiments also include or implement software, which can include one or more AI algorithms, to process data for predictive insights. Some embodiments can comprise a system comprising an onboard small language model (SLM) for real-time analysis and Software-as-a-Service (SaaS) models for cloud-based forecasting. Still other arrangements of various software and algorithmic components can be implemented in other embodiments, such as those which also interface or interact with other wearable smart devices, including smart phones, smart watches, smart glasses, smart jewelry (e.g., rings, bracelets, necklaces), and other wearable or portable smart or other computing devices.

The wellness-focused configuration can monitor general metrics, such as sleep duration, sleep quality or depth, heart rate (HR) trends, steps, restfulness score, average oxygen saturation, variations in blood oxygen, resilience (e.g., derived from HRV and stress data), and body temperature trends. Embodiments can be configured to avoid clinical thresholds to comply with the FDA's general wellness policy (21 CFR 880.9).

The medical-focused configuration can include validated diagnostic thresholds (e.g., AHI>5 for mild OSA, >100 g for concussions) and integrate with electronic health records (EHRs) for clinician review, requiring regulatory clearance. Both configurations can support at home customization (such as boil-and-bite fit), removable sensors for, e.g., upgradability, and a companion app for personalized recommendations.

Methods for manufacturing can include 3D printing trays with ±0.05 mm tolerance, injection molding sensor housings (Shore D 80-90), and robotic sensor assembly with ±0.1 mm accuracy, ensuring precision and compliance with ISO 13485 and IEC 60601-1. The disclosure is intended to broadly cover variations and future developments in sensor types and configurations, AI architectures, adjustment mechanisms, and operational modes, providing comprehensive non-invasive health monitoring.

Devices according to embodiments of the present disclosure address critical unmet needs in sleep health and sports safety, offering personalized, accurate, and comfortable solutions that improve quality of life and reduce long-term health risks. A WEAD according to one embodiment of the present disclosure is directed to snoring and sleep apnea by providing a mandibular advancement device (MAD) integrated with one or more sensors for the non-invasive management of snoring and OSA, offering precise measurement of oxygen saturation (SpO2), apnea-hypopnea index (AHI), heart rate, heart rate variability (HRV), respiration rate, sleep stages, and other health metrics. Another embodiment of the present disclosure is directed to concussion monitoring, serving as a concussive diagnostic tool for athletes in high-velocity sports, detecting impact forces, head acceleration, and physiological responses to enhance early detection and management of concussions. Devices according to embodiments of the present disclosure thereby can integrate health and safety while emphasizing the importance of addressing sleep health and concussion risks through advanced, AI-driven predictive analytics, real-time data feedback, and seamless integration with healthcare provider systems.

According to an aspect, a device of the present disclosure may include at least a wearable health metric technology to integrate mandibular advancement for OSA management with concussion monitoring for high-velocity sports, offering a multifunctional platform that addresses at least two distinct health and safety needs. The device can serve two distinct clinical applications using the same core platform with integrated health metric AI assistants and sensors.

For example, consider Patient A, a middle-aged individual with mild to moderate obstructive sleep apnea (OSA). For Patient A, the device can function as a mandibular advancement device (MAD), repositioning the jaw to maintain airway patency during sleep. The embedded sensors and other technologies can monitor airflow, oxygen saturation, and jaw positioning, enabling clinicians to assess treatment effectiveness and optimize therapy over time by accessing and reviewing collected data.

For Patient B, a young middle or high school athlete in a high-velocity contact sport, the device from the platform can operate as a smart mouth guard, capturing data related to impact forces, rotational acceleration, and head movement to detect potential concussive events. Both patients can benefit from real-time data and continuous monitoring and the device can deliver tailored AI-assisted insights—respiratory and sleep analytics for OSA management, and neurological impact data for sports injury prevention—highlighting its multifunctional value across two distinct health and safety needs.

Some embodiments of the present disclosure can include Advanced Sensor Integration, such as, for example, an “Oral Health AI Sensors” platform that combines research-grade sensors comprising one or more of PPG, temperature, accelerometers, gyroscopes, snore sensors, or any combination of these or other sensors, to provide comprehensive health and impact metrics, surpassing the capabilities of existing MADs and concussion-monitoring devices. In one example embodiment, the WEAD can comprise dual PPG units for cross-validation and modular click-in designs for upgradability, enabling at least some on-device AI or other data preprocessing.

As discussed, the WEAD can be a multifunctional oral appliance offered in two primary embodiments: a wellness-focused configuration for lifestyle monitoring, and a clinical or medical-focused version for diagnostic and therapeutic applications. Either or both configurations can comprise one or more biocompatible materials, including medical-grade polymethyl methacrylate (PMMA), polycarbonate, or reinforced polyetheretherketone (PEEK) for the outer shell, providing rigidity and durability (Shore D 80-90). The inner lining can comprise other (e.g., softer) materials such as medical-grade silicone, ethylene-vinyl acetate (EVA), or dual-polymer thermoplastic elastomer (TPE) for comfort and heat-moldable customization. The outer shell and inner lining can be sealed to IP67 standards, ensuring protection against oral moisture and sweat during extended wear up to 7 days, with submersion resistance up to 1 meter for 30 minutes.

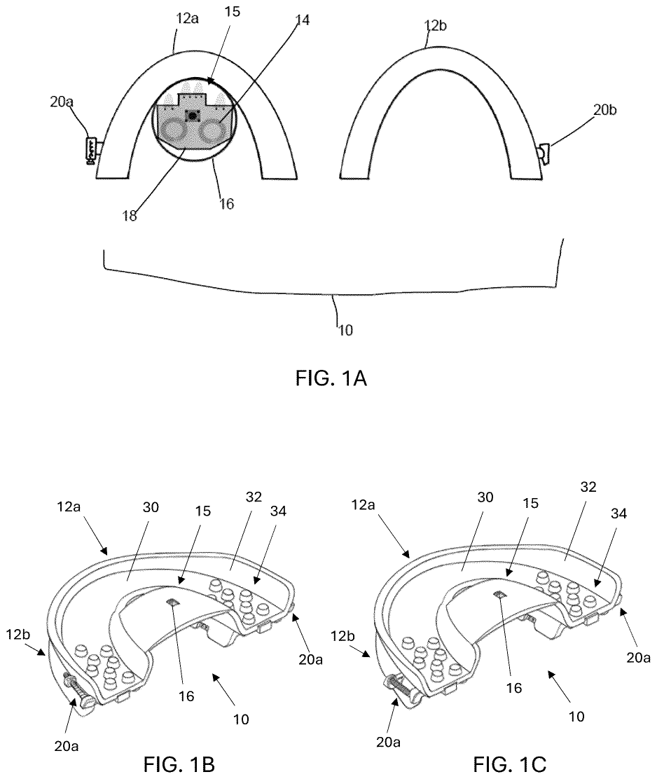

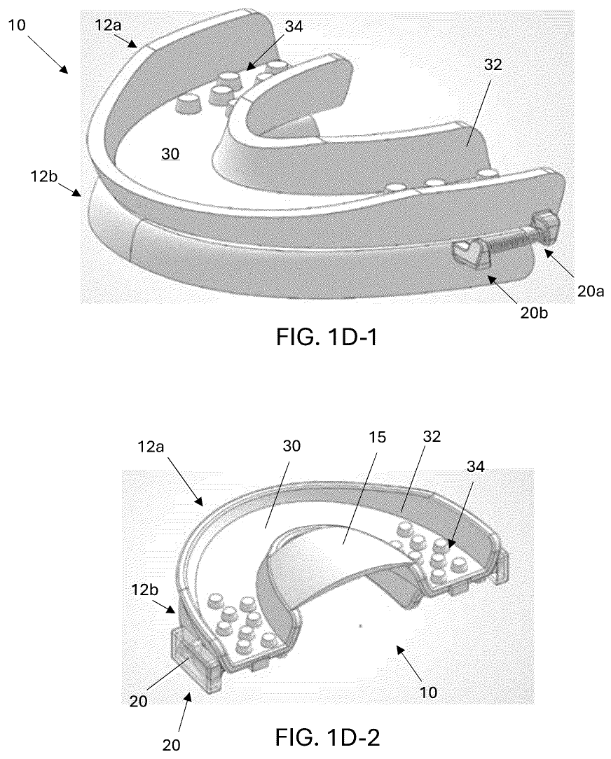

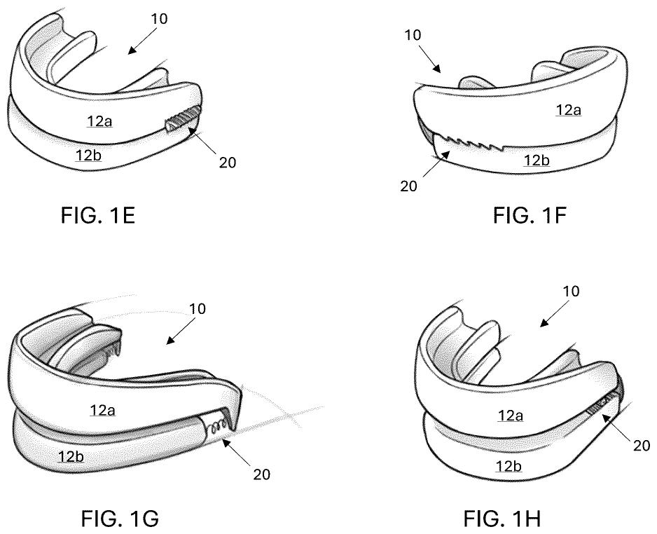

In an embodiment of the present disclosure shown in FIG. 1A, a WEAD 10 comprises a generally U-shaped body including a first 12a portion and a second portion 12b. First portion 12a can be configured to be worn on the top teeth and second portion 12b can be configured to be worn on the bottom teeth in various embodiments. Thus, each of first portion 12a and second portion 12b can comprise a tray, similar to an orthodontic retainer, aligner, biteguard, or mouthguard. As will be discussed, some embodiments, applications, or uses can comprise a single one of first portion 12a or second portion 12b (generally only first portion 12a in such an embodiment), while other embodiments can comprise both first portion 12a and second portion 12b, in which the two portions 12a, 12b are coupled or connected together for cooperative use.

In one embodiment, and referring also to FIG. 1B, first portion 12a can comprise a first body portion 30 and a second body portion 32. First and second body portions 30, 32 can be contiguous and comprise the same material(s) in some embodiments, while in other embodiments first and second body portions 30, 32 can be separately formed or comprise different materials.

In an embodiment of the present invention, WEAD 10 can be configured as an off-the-shelf Mandibular Advancement Device (MAD) in one or more sizes (e. g, small, medium, large) that can be adjusted by a healthcare provider for treating snoring and obstructive sleep apnea (OSA). In an embodiment WEAD 10 can be a custom-fitted oral appliance designed to treat snoring and OSA by advancing the lower jaw (mandible) forward during sleep, thereby maintaining an open airway. WEAD 10 can be worn in the mouth during sleep and is recommended for individuals with mild to moderate OSA or those intolerant to CPAP therapy. WEAD 10 can be fitted and adjusted in-clinic by healthcare providers (HCPs) to ensure precise mandibular positioning, enhancing efficacy and patient comfort.

As previously discussed, WEAD 10 can comprise one or more biocompatible, durable materials to ensure safety, comfort, and longevity. In some embodiments, first body portion 30 can comprise or include a smooth biocompatible material for user comfort, such as medical-grade silicone, ethylene-vinyl acetate (EVA), or a dual-polymer thermoplastic elastomer (TPE) blend. These formulations allow for heat-molding and improved conformability to the user's dentition, enhancing comfort, stability, and sealing during use.

Second body portion 32 can comprise a structured material to provide stability, such as medical-grade polymethyl methacrylate (PMMA), polycarbonate, or reinforced polyetheretherketone (PEEK), offering advanced rigidity, durability, and resistance to wear. These materials are widely used in dental and orthopedic applications due to their mechanical strength and long-term biocompatibility. Particular biocompatible materials can be chosen to prevent irritation or compression of vasculature and nerve endings, provide improved fit for a variety of palate shapes and sizes, enable boil-and-bite fitting, and for a variety of other reasons.

In one embodiment, an upper or additional layer can be included on or within first body portion 30 and can comprise a softer silicone, thermoplastic or other biocompatible material that ensures comfort and conformability to the oral cavity. Whether such a layer is used or not can be determined by an individual user, who can add or remove it for personalized fit or comfort.

In advanced versions, smart polymers with shape memory properties may be explored to enable self-adjusting fit and ease of insertion or removal, especially for dual-use applications across sleep and sports settings. The outer shell and inner lining are sealed to IP67 water-resistant standards, protecting against oral moisture and sweat during extended wear up to 7 days.

As depicted in FIGS. 1B and 1C, first body portion 30 can comprise one or more bite nubs 34 in some embodiments. Bite nubs 34 can provide a textured surface for teeth, improve fit stability, and provide other benefits.

Device 10 can include at least one open cavity 15 with a depth on its inner surface. Cavity 15 can be specially designed to custom fit to the maxilla while at the same time providing a housing for electronic components or sensors 14 configured to analyze and process biological signals. Sensors 14 can have a thickness that is less than the cavity's depth, ensuring a snug fit of WEAD 10. In an embodiment, sensors 14 having a thickness less than the depth of cavity 15 can be protected and encapsulated, such as by a durable epoxy material covering or other housing 16, or other suitable biocompatible material, ensuring both functionality and user safety.

According to an aspect, one or more of sensors 14 can be anatomically custom fit to the incisive papilla and incisive fossa. In some embodiments, one or more of sensors 14 can be arranged in or on housing 16, or some other portion of WEAD 10, to be positioned adjacent to a tooth line on a roof of an oral cavity. In any embodiment, it can be advantageous to have at least one sensor 14 arranged proximate a particular tooth or surface thereof (e.g., inner, outer, bite), an inner or outer gumline, the maxilla or palate, the tongue, the inner cheek or lip, or some other part of the mouth or oral cavity.

In still other embodiments, a plurality of sensors 14 are positioned in optimal positions in or on housing 16, or otherwise in or on WEAD 10, to optimize sensing and data collection for the particular sensor modality. Though in some embodiments all of sensors 14 are on first portion 12a, in other embodiments at least one sensor can be arranged on second portion 12b. In such an embodiment, sensors 14 can be arranged in a corresponding pair such a first sensor of the pair is on first portion 12a and a second sensor of the pair is on second portion 12b. A sensor pair can also comprise a sensor and a sensor marker or other object to be sensed by the sensor.

Other components, such as battery components and a microcontroller on a printed circuit board (PCB) 18, also can be arranged in cavity 15, such as in housing 16. In some embodiments, the battery can provide about 7 days of use with AI-activated low-power modes and inductive charging.

In other embodiments, sensors 18 and battery components can be housed between first portion 30 and second portion 32, sealed with permanent adhesive to prevent moisture ingress and ensure device integrity. In another embodiment, housing 16 is embedded between first portion 30 and second portion 32, with sensors 18 and battery components encapsulated in a biocompatible, moisture-resistant resin or sealed using permanent medical-grade adhesive to prevent fluid ingress and ensure long-term device integrity. Such a sealed compartment can includes a thin conformal coating over electronics for added protection against saliva and humidity.

In alternate configurations, sensor housing 16 can be modular or compartmentalized, allowing for removable or replaceable sensor and battery units, particularly in models designed for sports use where frequent charging or data retrieval is required. As shown in FIGS. 1B and 1C, housing 16 is strategically located in low-pressure zones (e.g., buccal flange or posterior palate) to minimize interference with jaw positioning and user comfort.

In an embodiment of WEAD 10, sensors 18 are integrated into an “Oral Health AI Sensors” platform, utilizing research-grade sensors to monitor and analyze metrics. Sensors 18 are strategically within first portion 12a just inside the tooth line on the roof of the oral cavity, abutting the soft tissue of the maxilla (e.g., incisive papilla and transverse palatine folds). Key sensor types include the following and any combinations thereof, but are not limited to:
Photoplethysmography (PPG) Sensors:
   Red and infrared LEDs measure blood oxygen levels (SpO2), heart rate, heart rate variability (HRV), and respiration rate during sleep.
   Green LEDs provide continuous heart rate monitoring, enhancing real-time data accuracy. For wellness, PPG data informs general trends like daily energy levels, average SpO2, and SpO2 variations; for medical, it detects desaturations with ±2% accuracy.
Accelerometers and Gyroscopes: Measure linear/rotational acceleration for concussion detection (>100 g linear or >4000 rad/s² angular, <1 ms resolution), or jaw positioning for MAD efficacy, with modular redundancy for upgradability.
Temperature Sensors: Track oral temperature variations (±0.1° C.) for circadian rhythm insights in wellness (e.g., correlating to sleep latency) and infection detection in medical use.
Snore Sensors: Microphone-based detection (>60 dB) for respiratory event flagging, integrated with AI for severity scoring, or piezo-based sensors.

In still other embodiments, wireless charging coils or inductive energy receivers are integrated within housing 16, enabling contactless recharging through first portion 30 or second portion 32. Sensor housing 16 itself can contain one or more modular, click-in sensors 18 (e.g., PPG, accelerometers, gyroscopes, temperature, snore sensors) and (in some embodiments) the battery. Particular sensors can be positioned on WEAD 10 and in cavity 15 and housing 16 for optimal sensing and data collection. For example, a PPG sensor for SpO2/HRV can be positioned on the buccal flange, and one or more accelerometers or gyroscopes for sensing jaw position/movement or impact can be positioned lingually. Dual PPG sensors can cross-validate SpO2 and HRV with ±2% accuracy in some embodiments, filtered for motion artifacts via accelerometer data (e.g., discard if accel >5 g).

According to one aspect, WEAD 10 can incorporate an adjustment mechanism 20. Adjustment mechanism 20 can allow for customization of WEAD 10 for various jaw sizes, ensuring a secure fit that prevents mandibular retraction. In an embodiment, adjustment mechanism 20 comprises a first component 20a on first portion 12a that operably engages with a second component 20b on second portion 12b.

For example, in the embodiment of FIG. 1D-1, first component 20a comprises a threaded female fastener, such as a worm screw, that operably engages with second component 20b that comprises a threaded male fastener, such as a clip, fastener or other suitable mechanism on second portion 12b. Operable engagement between first component 20a and second component 20b can enable an adjustable, secure fit between first portion 12a and second portion 12b. In embodiments, adjustment mechanism 20 is a dual adjustment mechanism to optimize mandibular advancement. In such an embodiment, adjustment mechanism 20 can include a titration screw embedded in device 10, allowing incremental adjustments as small as 0.1 mm, providing precise control over mandibular protrusion. Adjustment mechanism 20 can further include a sliding or ratchet mechanism 30 that enables in-clinic, virtual, or self adjustments ranging from, e.g., 0.1 mm to 3 mm in one visit or treatment to, e.g., 12 mm to 15 mm in the total course of treatment, to optimize airway opening while minimizing side effects such as jaw discomfort. Adjustment mechanism 20 enhances patient comfort and treatment efficacy, distinguishing WEAD 10 from existing MADs with less precise adjustment systems.

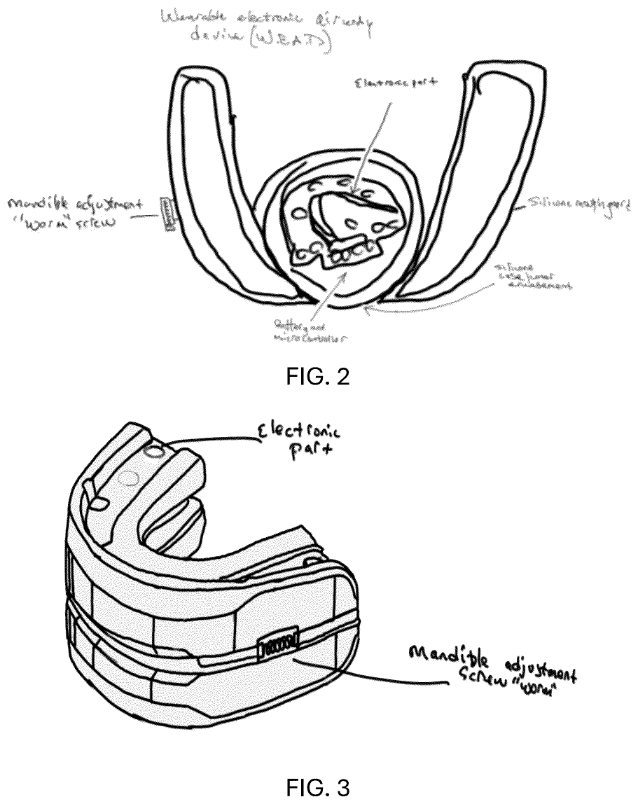

Some embodiments also can comprise a cover or housing 20c on or as part of adjustment mechanism 20. An example housing 20c is depicted in FIG. 1D-2. Housing 20c can be openable, partially removable, or fully removable in embodiments, in order to provide access to the other components 20a, 20b of adjustment mechanism 20. Housing 20c can provide one or more benefits, including helping to keep adjustment mechanism 20 clean and dry; or improving user comfort by providing a smoother, softer, or more ergonomic outer surface or configuration for adjustment mechanism 20.

An alternate embodiment of adjustment mechanism 20 is depicted in FIG. 1E. In this embodiment, at least one component of adjustment mechanism is at least partially recessed or embedded in first portion 12a or second portion 12b. In one particular example, part or all of adjustment mechanism 20 is substantially flush with a surface of first portion 12a or second portion 12b. Still other configurations and variations of these embodiments are also contemplated and considered to be part of this disclosure and within the scope of the claims.

Adjustment mechanism 20 can further incorporate a sliding, toothed, ratchet, or other two-part or cooperative engagement mechanism 30 between or among first portion 12a and second portion 12b, with an example depicted in FIG. 1F. Such an embodiment can allow in-clinic adjustments ranging from 0.1 mm to 3 mm in a single treatment session or more with consecutive treatment sessions, ensuring optimal airway opening while minimizing common side effects such as TMJ strain or bite misalignment.

In yet another embodiment depicted in FIG. 1G, adjustment mechanism 20 comprises a biasing element, such as a spring or elastic band. In some embodiments, the biasing element can be selected from a set of biasing elements such that different levels of tension can be provided between first portion 12a and second portion 12b according to the needs of a particular user. The biasing element can be interchangeable in some embodiments such that a tighter or looser engagement can be selected in a treatment session. In other embodiments, an adjustment mechanism can be included such that a single biasing element can be adjusted in situ.

FIG. 1H depicts a further embodiment, in which adjustment mechanism 20 comprises a magnetic coupling system with indexed positions for rapid chairside adjustments or a cam-lever system for controlled engagement with minimal tool use. In these embodiments, each of first portion 12a and second portion 12b can comprise one or more magnetic elements or sets of magnetic elements configured to cause selective and adjustable engagement between first portion 12a and second portion 12b.

In digitally-enabled versions, a micro-motor mechanism can be integrated to allow for remote or app-guided titration, providing data-driven adjustment protocols based on sleep metrics or impact data captured during use. These enhancements improve patient comfort, increase treatment adherence, and distinguish device 10 from conventional MADs with static or coarse adjustment methods. AI algorithms analyze sensor feedback during wear to recommend adjustments (e.g., +0.2 mm advancement if AHI>10 in medical mode, such as is depicted in the example embodiment of FIG. 2), supporting both diagnostic optimization and wellness habit formation.

In use, therefore, WEAD 10 (via adjustment mechanism 20) is configured to subtly reposition the mandible or tongue to maintain airway patency without causing strain on the palatal musculature. Ensuring even pressure distribution by WEAD 10 along the hard palate and dental arches can minimize user discomfort.

In one embodiment, adjustment mechanism 20 is configured as a dual adjustment system to optimize mandibular advancement with independent left-right calibration, accommodating asymmetries in jaw structure. The two adjustment mechanisms 20 can be the same (e.g., mated worm screw and clip) or different. In another example, WEAD 20 can comprise a plurality of adjustment mechanisms 20, with more than one being provided on one or both sides of WEAD 20. This can enable opposing biasing arrangements for situations in which a larger range of repositioning is desired or for some other reason. In still other embodiments, a plurality of adjustable connectors 20 can be included in order to improve fit or comfort, or for some other reason.

By integrating sensors 14, WEAD 10 can be used to monitor essential respiratory parameters, providing valuable data on the user's condition which can include but is not limited to: breathing regularity, average oxygen saturation, total sleep, efficiency of sleep, restfulness, Rapid Eye Movement (REM), deep sleep, latency and timing, resting heart rate, heart rate variability, body temperature, and respiratory rate.

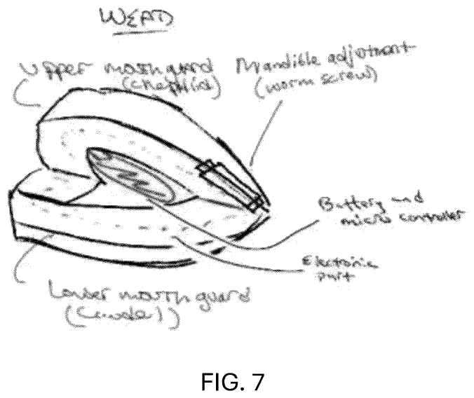

Alternate embodiments and features also are depicted in FIGS. 2-7.

### Wellness-Focused Embodiments

Wellness-focused configurations, such as the embodiment of FIG. 2, can comprisea boil-and-bite mouthguard version of WEAD 10 that can be provided in a range of sizes (e.g., small, medium, large sizes; pediatric/child and adult ranges) or as a single-piece upper mouthguard (such as is depicted in FIG. 4) for non-diagnostic monitoring such that it can be marketed and sold over the counter. One embodiment of this mouthguard omits the mandibular adjustment mechanism 20, with intended uses geared towards protection in high-velocity sports and general health metrics inspired by leading wearables like OURA RING GEN4, GOOGLE PIXEL WATCH, and APPLE WATCH.

In other words, the embodiments of, e.g., FIGS. 2 and 4 can comprise either a boil-and-bite configuration with sensors for concussion detection and management in sports or other activities, or a configuration comprising first portion 12a for other non-diagnostic (e.g., non-concussive) applications or others. Therefore, in some embodiments, embodiments of WEAD 10 can provide one or more of:
Sleep Duration Tracking: Tracks total sleep time (target 7-9 hours)
Heart Rate Trend Monitoring: Monitors average and resting HR (60-100 bpm
Steps and Activity Level Tracking: Counts daily steps (e.g., >5,000 target) and movement
Restfulness Scoring: A 0-100 score based on HRV (SDNN >50 ms) and deep sleep (>13%)
Average Oxygen Saturation: Measures nightly SpO2 trends (e.g., 95-100% normal) or variations
Variations in Blood Oxygen: Tracks SpO2 fluctuations (>5% alerts)
Resilience: Derived from HRV, sleep quality, and activity (e.g., stress response trends)
Body Temperature Trends: Monitors ±0.1°C variations for circadian rhythm insights. The app can generate weekly/monthly health reports summarizing these metrics, offering non-diagnostic recommendations (e.g., “Your resilience score of 75 suggests good recovery—maintain activity levels”). AI-driven insights avoid clinical thresholds (e.g., no AHI or SpO2 <88% alerts) to comply with FDA's general wellness policy (2016/2019).

### Medical-Focused Embodiment

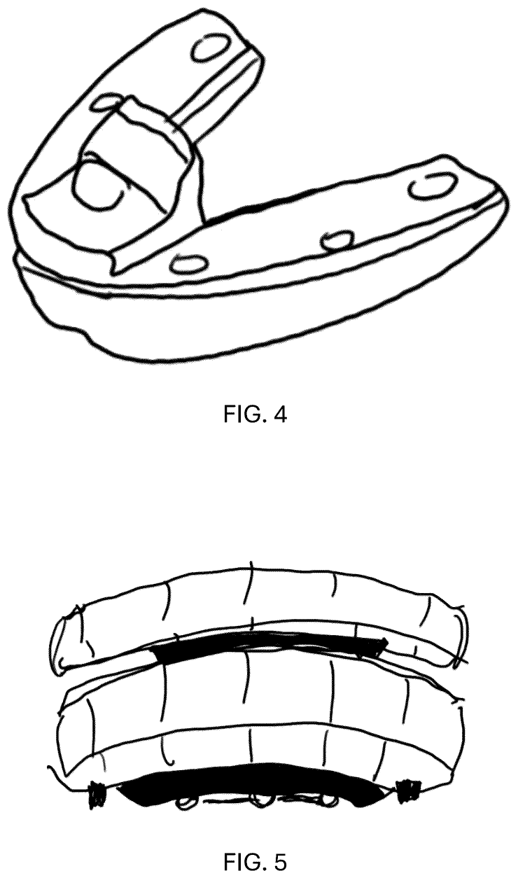

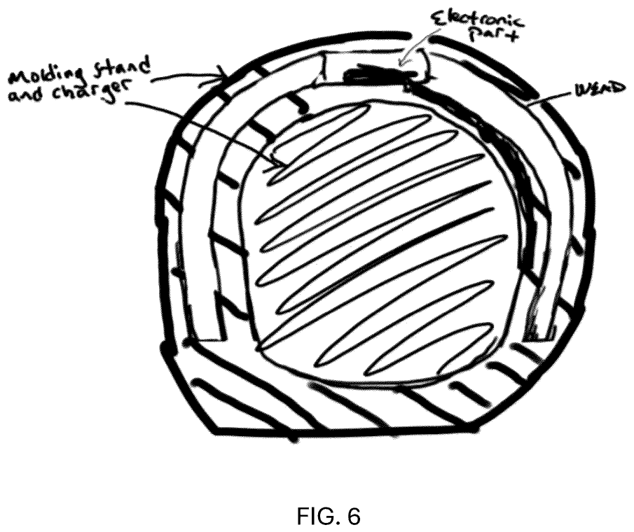

One embodiment of the medical-focused configuration (such as is depicted in FIGS. 3 and 4, among others) includes the adjustment mechanism 20 discussed previously. for OSA management. Another embodiment can comprise a one-piece mouthguard for concussion diagnostics. These embodiments can monitor or measure:
OSA Metrics: AHI (events/hour), SpO2 desaturations (<88% flags hypoxia), HRV, respiration rate, snoring intensity (>60 dB), and sleep stages, impact acceleration, and HR spikes, with validated thresholds for diagnosis (e.g., AHI>15 for moderate OSA).
Concussion Metrics: Linear/rotational acceleration (>100 g or >4000 rad/s², <15 ms duration), impact count/location, and physiological responses (e.g., HR spikes>20 bpm). This version integrates with electronic health records (e.g., Epic) via HL7/FHIR for clinician review, supporting mandibular titration (e.g., +0.2 mm if AHI>10).

In either or both wellness-and medical-focused embodiments, additional variations also can be included for acute vs. chronic condition monitoring:
Acute (e.g., concussion): Alerts for impacts >100 g or >4000 rad/s² (<15 ms) via app notifications and cloud-based SCAT-integrated platforms.
Chronic (Sleep/Lifestyle): RNNs predict OSA exacerbation (90% accuracy) or CTE risk (>5000 g cumulative), with app-based lifestyle recommendations (e.g., reduce high-risk activities if >20 impacts/week).

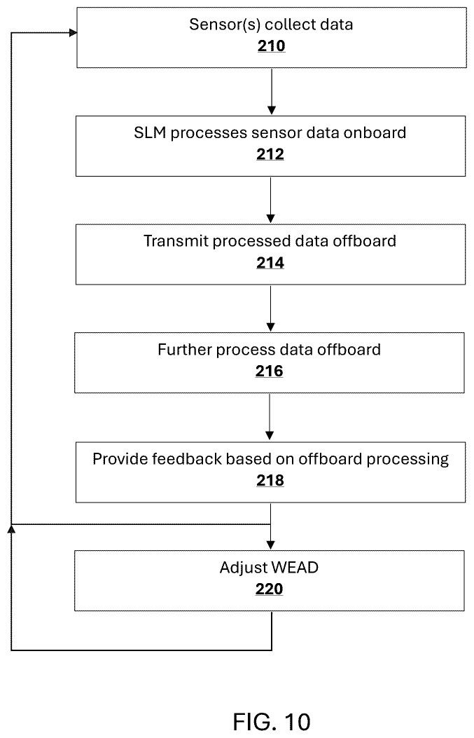

A flowchart of an example method of the disclosure is depicted in FIG. 10. At 210, sensor(s) 16 collect data while WEAD 10 is worn by a user.

At 212, some or all of the collected sensor data is processed onboard WEAD 10 by a small language model (SLM). This can include feature extraction (e.g., PPG waveforms) or real-time analysis (e.g., sleep stage probabilities).

The processed data is then transmitted offboard WEAD 10 at 214. This can comprise using BLUETOOTH LOW ENERGY (BLE) or another short-range wireless transmission methodology to transmit the processed data from WEAD 10 to a mobile or smart device running a companion app, as discussed elsewhere herein. In some embodiments, wired or contact-based transmission methodologies can be used additionally or instead of BLE.

Additional data processing then can be performed offboard WEAD 10 at 216. This can be done on the smart device, such as via a related app, website, or platform, or operation 214 can further comprise transmitting some or all of the data to the cloud for additional processing. This can include maintaining HIPAA-compliance and using encryption, with further processing, refinement, and analysis of the data performed. For example, the additional processing (regardless of where performed) can include advanced data processing with RNNs/LSTMs, which can provide trend forecasting such as OSA risk and predictive analysis. From this, predictive analyses can be performed and used to generate insights, reports, alerts, treatments steps or plans, or other feedback.

This feedback can be provided at 218, directly to a user via the aforementioned app or on a website, or to a clinician or medical provider (e.g., via a dedicated WEAD app or platform for providers, in EPIC, or in some other way). This feedback can include progression to 220 for adjustments to WEAD 10 or maintenance wear, with the method returning to 210 for further data collection.

### Concussion Management Embodiments

In yet another embodiment of the present disclosure, WEAD 10 can be directed to a wearable technology for concussive diagnosis in high-velocity sports. WEAD 10 can be configured as an upper mandibular device, worn on the upper teeth during high-velocity sports activities. WEAD 10 can be custom fitted to ensure comfort, conformability, and secure placement, making it suitable for athletes in contact and non-contact sports.

WEAD 10 can be constructed from durable, biocompatible materials to withstand impact forces while ensuring user comfort. WEAD 10 can include an outer portion as discussed elsewhere herein comprised of medical-grade acrylic (PMMA), or any other suitable biocompatible material that provides rigidity and impact resistance. In an aspect, WEAD 10 can include an second portion comprised of a softer silicone, thermoplastic or other biocompatible material that ensures comfort and conformability to the oral cavity. Suitable materials can include medical-grade silicone for its flexibility and hypoallergenic properties, ethylene-vinyl acetate (EVA) for its cushioning effect and moldability, or thermoplastic elastomers (TPEs) for their unique ability to blend softness with durability. Advanced versions may utilize dual-laminate systems, combining a rigid PMMA or PEEK outer layer with a heat-moldable EVA or TPE inner core, allowing chairside customization and improved fit. In some embodiments, shape memory polymers (SMPs) or pressure-sensitive hydrogels may be integrated to allow dynamic conformability or adaptive fit in response to body heat or bite pressure, further enhancing retention and user experience. These material combinations ensure the device performs reliably in both therapeutic and high-impact sports settings.

WEAD 10 can comprise a novel sensor system to detect and analyze impact forces, head movement, and physiological responses. Sensors can be arranged substantially as discussed with respect to other embodiments, including abutting the soft tissue of the maxilla, to ensure accurate data collection. Key sensor types and combinations may include, but are not limited to:
Accelerometers and Gyroscopes: Measure linear and rotational acceleration of the head, detecting impact forces and head movement.
Physiological Sensors: Monitor heart rate and potential neurological markers, providing insights into physiological responses to impact.
   The sensors can be integrated with microcontrollers, memory, and proprietary AI algorithms to process raw data into actionable metrics, transmitted via Bluetooth to smartphones or cloud platforms for analysis. Thus, according to an aspect of the present disclosure, WEAD 10 can provide real-time monitoring and analysis of concussion risk, offering at least the following capabilities, and any combination thereof:
Impact Detection: Measures impact force, acceleration, and head movement, providing data on the magnitude and direction of impacts.
Physiological Monitoring: Tracks heart rate and other physiological responses, identifying potential signs of concussion.
Risk Assessment: Proprietary AI algorithms analyze sensor data in real-time, generating an overall risk score to supplement existing sideline assessments.

WEAD 10 can enhance the accuracy and objectivity of concussion detection, addressing the limitations of subjective tools and delayed assessments. WEAD 10 can further include Bluetooth or other wireless connectivity such that WEAD 10 can connect wirelessly to smartphones and smart devices, enabling real-time data transmission and feedback. In an embodiment, WEAD 10 can include a novel application interface that provides real-time feedback for coaches, medical staff, parents/guardians, and athletes, including impact data, physiological metrics, and risk scores. This data supports informed decision-making, such as return-to-play protocols, enhancing athlete safety. Access to the application interface may be available via a monthly subscription, ensuring ongoing updates and support. All data management and storage systems are HIPAA-compliant, ensuring athlete privacy and security.

WEAD 10 can serve as an adjunct to current concussion evaluation protocols, offering objective, real-time data to enhance early detection and management of concussions. By integrating advanced sensors and AI analytics, WEAD 10 can support safer participation in high-velocity sports, reducing the risk of long-term neurological damage and improving athlete outcomes.

### AI and Sensor Enablement

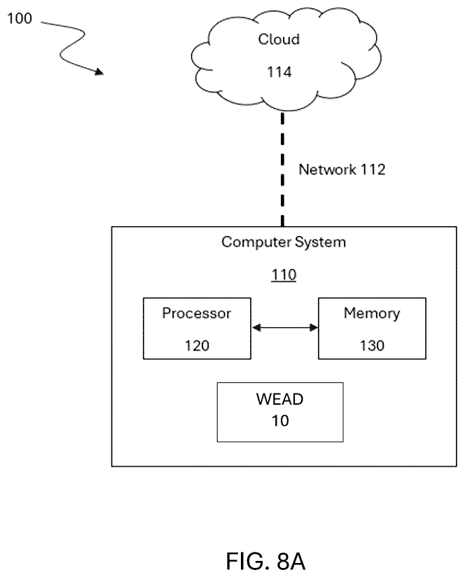

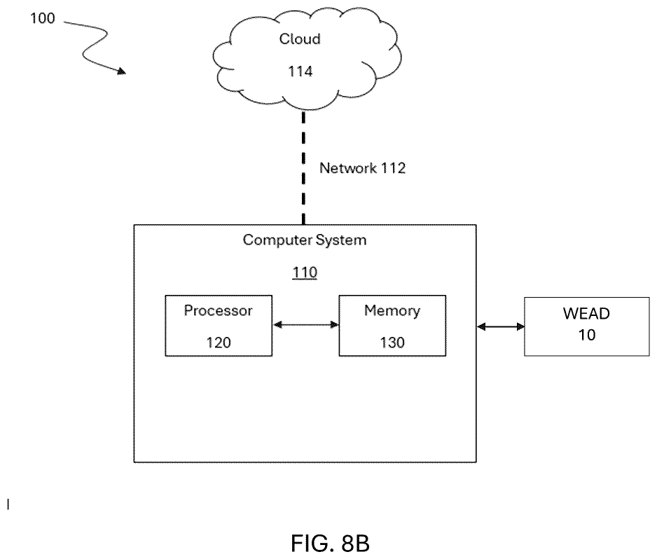

FIGS. 8A and 8B are block diagrams of a system 100 according to at least one embodiment of this disclosure. System 100 comprises a computer system 110, which can be or include WEAD 10.

In the simplified conceptual view of FIG. 8A, computer system 110 comprises at least one processor 120 and memory 130 and WEAD 10. In the embodiment of FIG. 8B, WEAD 10 cooperates and communicates with computer system 110.

Embodiments of the systems and methods (and other components or processes, including algorithms) discussed herein can either operate on or in conjunction with at least one processor and memory, such as processor 120 and memory 130. For example, in one embodiment processor 120 and memory 130 can be partially or completely cloud-based 114, with user interaction facilitated remotely and via the internet or another network 112, an application programming interface (API) presented on a computing device, such as a computer, laptop, smart phone, tablet, smart watch or other wearable, or other device or system, or some other computing device portal.

In still other embodiments, computer system 110 can be a local computing device communicatively coupled via a network 112 to other computer systems. For example, network 112 can be a local area network (LAN), wide area network (WAN), or the internet, with cloud 114 in FIG. 8 representing at least one computer or computing device. Virtually any arrangement of computers and computing devices, local or remote, coupled via a public or private network, or any combination of these variations, can be used in various embodiments, with the various illustrative examples given herein not being limiting.

Processor 120 or any of the other systems or components discussed herein with respect to computer system 110 can be or work in cooperation with any programmable device (or system or network of devices) that accepts digital data as input, is configured to process the input according to instructions or algorithms, and provides some output as a result. In one example embodiment, processor 120 can be a central processing unit (CPU) or a microcontroller or microprocessor (or group of microcontrollers or microprocessors) configured to carry out the instructions of a computer program or software, firmware, another type of code or coding, or a combination thereof. Processor 120 is therefore configured to perform at least basic arithmetical, logical, and input/output operations.

Processor 120 includes or is communicatively coupled with memory 130 or other digital storage, which can comprise volatile or non-volatile memory as required by processor 120 to not only provide space to execute the instructions or algorithms, but also to provide the space to store the instructions themselves. In various embodiments, volatile memory can include random access memory (RAM), dynamic random-access memory (DRAM), or static random-access memory (SRAM), for example. In embodiments, non-volatile memory can include read-only memory, flash memory, ferroelectric RAM, hard disk, or optical disc storage, for example.

In some embodiments, system 100 of FIGS. 8A and 8B (whether as part of memory 130, in communication with memory 130, or separately (e.g., in cloud 114) includes or interacts with various databases. These databases can include one or more system databases, SQL databases, vector databases, curated or proprietary databases, data management systems, or other data stores or repositories.

The foregoing examples in no way limit the types of processing hardware or systems, or memory hardware or systems, that can be used in various embodiments, as these examples are given only by way of example and are not intended to limit the scope of the present disclosure or embodiments discussed herein. For example, both processor 120 and memory 130 can be cloud-based but nevertheless comprise physical infrastructure on a server or server farm, consistent with previous disclosure herein. Furthermore, computer system 110 can be or represent any computer system that forms part of, or interacts with, the various computers and computer systems described herein. For example, one embodiment can comprise a plurality of computer systems 100. All of this applies as well to other systems and computer systems described herein.

Machine learning (ML) is a field of artificial intelligence (AI) in which algorithms can be trained by and learn from data in order to generalize new or unseen data. ML can be or include a large language model (LLM), which is an AI model (in particular, an artificial neural network, or ANN) that can understand and generate language. Conventional examples of LLMs include GPT, GEMINI, LLAMA, CLAUDE, and others. AI models also can include small language models (SLMs), which are compact natural language processing models designed to operate efficiently in resource-constrained environments (such as onboard WEAD 10). While examples discussed herein may use or apply ML, LLMs, and SLMs, other types of AI may be used in embodiments of the systems and methods of this disclosure without being limited by any particular mention or example related to ML used herein. Furthermore, examples or types of ML or AI or future iterations of these technologies and techniques not yet known but nevertheless relevant to embodiments herein and falling within the scope of the disclosure and claims can be relevant to or used with embodiments of this disclosure, particularly given the rapid pace of advancement of AI and ML at this time and the foundational understanding those of ordinary skill in the art will have with respect to these techniques as they evolve.

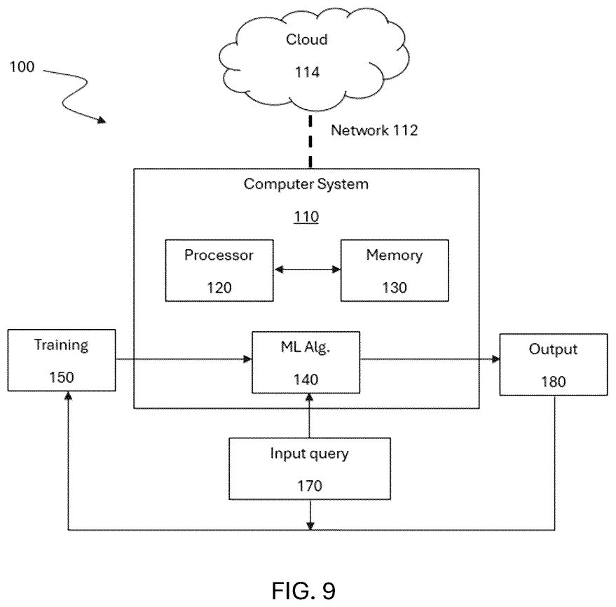

ML algorithms and other AI techniques operate on, or are implemented at least in part by, system 100, such as is depicted in system 200 of FIG. 9. Computer system 110 can have virtually any suitable form, such as mainframe, mini, micro, super, server, or any combination of these or other types of computer systems. Moreover, computer system 110 can be virtual, cloud-based, or physical. In other words, the particular implementation of computer system 110 can vary. As depicted in FIGS. 8A and 8B, in some embodiments WEAD 10 can be part of system 110 (FIG. 8A), whereas in others WEAD 10 communicates with computer system 110 (FIG. 8B).

Computer system 110 further comprises, or is communicatively coupled to interact with, an ML algorithm 140. Though ML algorithm 140 is depicted as a separate component in FIG. 2, those skilled in the art will recognize that ML algorithm 140 can be stored in memory 130 and executed by processor 120 in use and operation. In other embodiments, ML algorithm 140 can be stored remotely from computer system 110 or executed in conjunction with processors or memory separate from computer system 110. For example, ML algorithm 140 can reside on cloud 114 or a device coupled to computer system 100 via network 112. As such, FIGS. 8 and 9 can be viewed as functional or conceptual diagrams rather than as strict structural representations.

A base requirement for ML algorithm 140 is that it is an algorithm that can be trained by and learn from data in order to generalize new or unseen data. Though described as a machine learning algorithm, ML algorithm 140 can be any type of artificial intelligence (AI) or neural network (NN) algorithm.

In various embodiments, ML algorithm 140 receives (or uses, or processes) training data and information (“training”) 150. As depicted in FIG. 9, training 150 is received from outside of computer system 110, but in other embodiments training 150 can be stored by memory 130 and provided therefrom to ML algorithm 140, or provided to ML algorithm 140 in some other way. In some embodiments, training 150 can comprises proprietary data and information. In this way, ML algorithm 140 is trained with information as part of training 150 that is not generally publicly available, cannot be generated by a computer alone, and may be expertly produced or assembled with a goal of maximizing the effectiveness of ML algorithm 140 with respect to a particular use or application. Furthermore, training ML algorithm 140 with proprietary data and information separates and elevates the performance and efficacy of ML algorithm 140 as compared with conventional ML algorithms or AI systems that are commonly available via the internet but merely apply public information scrubbed from the internet. In addition to proprietary data and information, training 150 also can include selective publicly available information.

Some embodiments can implement AI processing using a hybrid SLM/SaaS architecture (see, e.g., FIG. 10):
SLM (On-Device): Distilled transformer (10-50 M parameters) for real-time tasks like sleep stage classification. SLMs preprocess raw sensor data (e.g., PPG for SpO2, HRV, respiration) using convolutional neural networks (CNNs) for feature extraction, achieving apnea event detection accuracies of 67.03% to 98.60% (median 89.66%). For concussion monitoring, SLMs classify impact events (>100 g or >4000 rad/s²) with physiological correlations (HR spikes>20 bpm). Example pseudocode: pythondef classify_event(sensor_data):
if accel_data>impact_threshold: #e.g., 100 g for concussion
return “acute_impact_detected”
features=cnn_extract(ppg_data) #Extract SpO2/HRV features
return slm_model.predict(features) #Outputs “apnea_event” or “normal”
SaaS (Cloud): LSTM networks for long-term predictions (e.g., OSA exacerbation risk at 90% accuracy, CTE risk>5000 g cumulative impacts). Example: pythondef forecast_risk(historical_data):
lstm_model=load_saa_model(‘lstm_risk’)
trends=lstm_model.fit(historical_data).forecast(7) #7-day prediction
if trends[‘impact_cumulative’]>5000: return “CTE_risk_high”
return trends #Outputs e.g., “OSA_exacerbation_90% probability”

Training can use anonymized datasets (>1,000 sleep cycles, 500 sports sessions), and federated learning can ensure privacy-compliant model updates. This differentiates from conventional approaches like deep learning for OSA (95% accuracy, abdomen-worn) or AI in sports injury prevention.

### Mobile Application Description

A WEAD Companion App can be provided to accompany WEAD 10, available for iOS and Android. Such an app can provide an AI-enhanced personalized health optimization platform for WEAD 10, differentiating wellness and medical use cases. Compliant with HIPAA via AES-256 encryption, role-based access, and audit trails, the app can use a “freemium” model: free basic features and premium (subscription) for advanced AI insights.

In an embodiment of the present disclosure, WEAD 10 can include Bluetooth or other wireless connectivity (as discussed elsewhere herein) such that WEAD 10 can be connected wirelessly to smartphones and smart devices, enabling real-time data transmission and feedback. According to an aspect of the present disclosure, the “Oral Health AI Sensing” platform can provide detailed analytics for both patients and healthcare providers (HCPs), including SpO2 trends, AHI scores, HRV, resting heart rate, and sleep stage breakdowns. This data supports clinical decision-making, treatment optimization, and patient education, enhancing overall health outcomes and quality of life. Access to the application interface may be available via a monthly subscription, ensuring ongoing updates and support. All data management and storage systems are HIPAA-compliant, ensuring patient privacy and security.

### Manufacturing Methods

The methods of manufacturing various embodiments of WEAD 10 and other components of this disclosure can involve precise molding techniques, quality checks, and encapsulation processes to create a durable, safe, and user-friendly product. According to one aspect of the present disclosure, the structural fit of WEAD 10 can be accomplished using precise 3D imaging, including but not limited to CBCT scans to model palatal and alveolar topography accurately so as to avoid impinging on soft tissue or key neurovascular structures. Adjustable components allow fine-tuning based on patient feedback to address individual anatomical variations and airway requirements. This integrative approach ensures that the custom-fit WEAD 10 is both effective and comfortable for treating snoring and mild to moderate obstructive sleep apnea.

WEAD 10 supports digital oral scanning for custom fits, with AI-driven algorithms optimizing advancement of adjustment mechanism 20 (0.1-3 mm) based on anatomical data. BLE 5.0 enables HL7/FHIR data export to EHRs (e.g., Epic) for medical diagnostics and API integration with sports platforms (e.g., Catapult) for wellness performance tracking. Intraoral scans are imported into CAD software for tray design, with AI simulating mandibular movement to minimize TMJ stress. Boil-and-bite embodiments use thermoplastic materials for at-home molding and can include embedded QR codes or other easy-access features to access app-guided fitting.

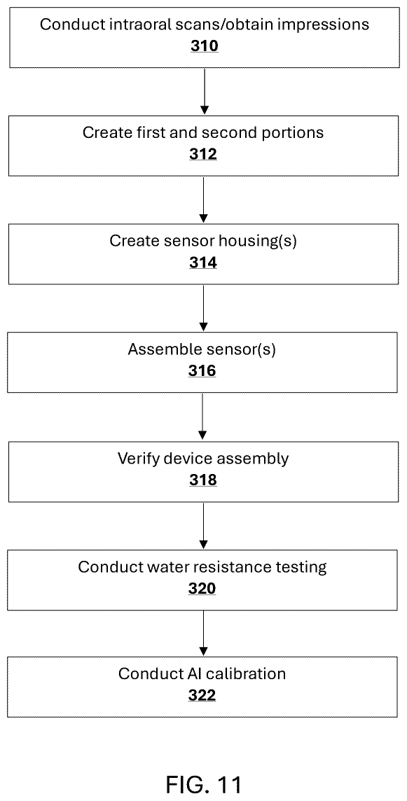

A method for manufacturing WEAD 10 is depicted in FIG. 11. At 310, intraoral scans of a patient can be conducted, as a way to take digital impressions for custom design of WEAD 10. In some embodiments, traditional (non-digital) impressions can be taken additionally or instead.

At 312, first portion 12a and second portion 12b are created. In one embodiment, this comprises 3D printing first portion 12a and second portion 12b from medical-grade PMMA (or another suitable material). Advantageously, this printing can be performed with with ±0.05 mm tolerance to provide a precise, patient-specific fit for comfort and treatment/use efficacy.

At 314, injection molding of the sensor housing(s) is performed. In some embodiments, the sensor housing(s) can be created in ways other than injection molding. In one embodiment, the sensor housing comprises a biocompatible polycarbonate (Shore D 80-90), which provides a durable, sufficiently rigid enclosure for the sensor(s) in a human oral environment.

Robotic, hand, or other assembly of the sensor(s) then can be performed at 316. Robotic assembly to provide ±0.1 mm accuracy can be advantageous. This provides precise placement of the sensor(s), such as ensuring a PPG sensor is sufficiently placed relative to the palate when WEAD 10 is worn, to avoid errors, provide for desired placement for data collection, and ensure reliability of WEAD 10.

At 318, post-assembly verification of WEAD 10 can be performed. This can include finite element analysis (FEA) and simulation of the structural integrity of WEAD 10, such as for impacts under 100 g. This verification can be helpful to ensure WEAD 10 can withstand the forces that can occur in high-velocity sports, maintain structural integrity if dropped by a user, and otherwise maintain structural integrity during expected use without failure to optimize the design.

Water resistance testing also can be performed at 320. This can include WEAD 10 undergoing IP 67 water resistance testing (i.e., submersion up to 1 meter for 30 minutes) to confirm protection against saliva, sweat, and other fluids for use in the mouth and during sports or other activities.

At 322, AI calibration for baseline user fitting can be performed, ensuring compliance with ISO 13485. This can include initializing the AI with user-specific data for accurate analytics.

Throughout this method, various automated quality control operations can be performed, such as using machine vision for sensor alignment and material integrity checks, differentiating from manual assembly in conventional approaches.

Other manufacturing methodologies can be used in other embodiments, without limitation.

### Regulatory Compliance

Embodiments of WEAD 10 can provide HIPAA compliance through any or all of end-to-end encryption (AES-256), role-based access controls, audit trails, and de-identification for analytics. Data can be shared only with explicit user consent, and providers can be given access via secure portals only in various embodiments.

In embodiments in which app access is provided, various app subscriptions can be offered and can include premium features like custom reports and AI insights. Data resale can be limited or prohibited. The SaaS component can provide limited free or subscription-based guidance.

The 21st Century Cures Act (2016) supports innovation, particularly for SaMD. The wellness-focused embodiment of WEAD 10 aligns with Section 3060 exemptions for low-risk devices, while the medical version requires 510(k) clearance for diagnostic claims. ISO 13485 and IEC 60601-1 compliance ensures manufacturing and electrical safety, supporting global markets (e.g., EU MDR).

The Wearable Electronic Airway Device (WEAD) is a groundbreaking wearable health metric technology that addresses critical unmet needs in sleep health, sports safety, and wellness. By integrating mandibular advancement, advanced sensors, AI analytics, Bluetooth connectivity, and extended battery life, the WEAD offers a personalized, accurate, and comfortable solution for snoring and OSA management, as well as concussion monitoring in high-velocity sports. The device's dual-application design, proprietary sensor system, and AI-driven analytics distinguish it from existing technologies, positioning it as a transformative tool in the wearable medical device market.

Embodiments of WEAD 10 are configured to provide a personalized, accurate, and comfortable alternative to existing OSA treatments, addressing the limitations of CPAP machines and traditional oral appliances. By integrating advanced sensors and AI analytics, device 10 provides actionable insights into OSA severity, treatment efficacy, and overall health, supporting early intervention and long-term management of sleep disorders. Accordingly, numerous improvements and advantages are provided by embodiments of this disclosure. For example, embodiments can provide the following:

### Real-Time Data Visualization

Wellness Use Case: Intuitive dashboard displays non-diagnostic metrics, including sleep duration (7-9 hours target), HR trends (60-100 bpm), steps (>5,000/day), restfulness score (0-100, based on HRV SDNN>50 ms and deep sleep>13%), average SpO2 (95-100% normal), SpO2 variations (>5% alerts), resilience (HRV/stress-derived), and body temperature trends (±0.1°C). Gamified visuals can encourage behavior modification, e.g., “Your low REM (18%) correlates with <30 min evening unwind—try a 10-min meditation streak.” Weekly/monthly health reports summarize trends, e.g., “Your resilience score of 75 suggests good recovery—maintain activity.” No clinical thresholds ensure FDA compliance.
Medical Use Case: Clinical dashboards display AHI, SpO2 (<88% flags hypoxia), HRV, impact metrics (>100 g events with location mapping), and snoring intensity (>60 dB). Provider portals enable secure sharing, e.g., “AHI 12.5 indicates moderate OSA—recommend 1 mm advancement.”
Interface: Color-coded, swipeable tabs with interactive line graphs (e.g., SpO2 trends, FIG. 12); dark/light modes; voice-over accessibility.

### Personalized Insights and Recommendations:

Wellness Use Case: SLMs detect trends (e.g., caffeine-sleep latency links), suggesting, “Reduce post-3 PM intake to gain 15% more REM.” Lifestyle logs (e.g., steps, diet) enable correlations, with badges for streaks (e.g., 7-day sleep goals).
Medical Use Case: SaaS RNNs predict risks (e.g., 90% OSA exacerbation accuracy), generating reports: “Cumulative impacts 4500 g/season elevate CTE risk—prescribe rest protocol.” Threshold-based alerts for acute events.
Interface: “Insights Hub” tab with push notifications; gamified wellness goals; exportable PDFs for medical compliance.
   Integration with External Systems:
Wellness Use Case: Syncs with, e.g., APPLE HEALTH/GOOGLE FIT for aggregated data (e.g., steps vs. HRV).
Medical Use Case: HL7/FHIR to EHRs (Epic/Cerner); APIs to sports tools (CATAPULT).
Interface: “Connect” tab with one-tap setup and consent prompts.

### Customization and Alerts:

User-set thresholds (e.g., wellness: SpO2<92%**; medical: **>100 g). Offline storage (7 days); auto-sync.
Interface: “Settings” for units/preferences; “Nightly Summary” emails.

### Educational Content and Support:

Tutorials on fitting/maintenance; AI chatbot for queries (e.g., “How to interpret resilience?”).
Interface: “Learn” tab with videos; multilingual.
   Technical Architecture: BLE 5.0 for low-latency; on-device SLMs for privacy; AWS cloud for SaaS. Onboarding via QR-pairing (<2 min).

Accordingly, this disclosure includes embodiments according to the following clauses:

Clause 1: A wearable electronic airway device (WEAD) comprising: a first portion comprising: a housing comprising at least one sensor, a power source, wireless communications circuitry, and a processor comprising a small language model (SLM), and a first component of an adjustment mechanism, a second portion comprising a second component of the adjustment mechanism, the second component configured to selectively engage with the first component to adjustably couple the first portion and the second portion, wherein, in use by a human user having upper teeth and lower teeth, the first portion is configured to be worn on the upper teeth such that the at least one sensor is positioned adjacent to a tooth line on a roof of an oral cavity, the lower portion is configured to be worn on the lower teeth, and mandibular advancement or retraction is provided by adjusting a position of the second portion relative to the first portion using the adjustment mechanism.

Clause 2: The WEAD of Clause 1, wherein the housing is removable from the first portion and the WEAD.

Clause 3: The WEAD of Clause 1 or Clause 2, wherein the at least one sensor comprises at least one of the group consisting of: a photoplethysmography (PPG) sensor, an accelerometer, a gyroscope, a microphone, a temperature sensor, and a piezo sensor.

Clause 4: The WEAD of any of Clauses 1-3, wherein the power source is a rechargeable battery.

Clause 5: The WEAD of any of Clauses 1-4, wherein one of the first component or the second component of the adjustment mechanism comprises a titration screw, and the other of the first component or the second component comprises a ratchet.

Clause 6: The WEAD of Clause 5, wherein the titration screw is incrementally adjustable relative to the ratchet, and wherein a recommended amount of incremental adjustment for the human user is provided at least in part by the SLM based on sensor data from the at least one sensor.

Clause 7: A system comprising: the WEAD of any of Clauses 1-6 1; and an application operating on a computing device, the computing device configured to receive SLM-processed sensor data from the WEAD via the wireless communications circuitry.

Clause 8: The system of Clause 7, further comprising a cloud-based computing system configured to receive the SLM-processed sensor data from the application operating on the computing device.

Clause 9: The system of Clause 7 or Clause 8, wherein at least one of the computing device or the cloud-based computing system is configured to further process the SLM-processed sensor data to obtain further processed sensor data.

Clause 10: The system of Clause 9, wherein at least one of the computing device or the cloud-based computing system is configured to provide a recommended adjustment of the adjustment mechanism to change the position of the second portion relative to the first portion based on at least one of the SLM-processed sensor data or the further processed sensor data.

Clause 11: A method of managing a wearable electronic airway device (WEAD), the WEAD comprising a first portion and a second portion, the first portion including at least one sensor, a processor comprising a small language model (SLM), and a first component of an adjustment mechanism and the second portion comprising a second component of the adjustment mechanism, the second component configured to selectively engage with the first component to adjustably couple the first portion and the second portion, the method comprising: receiving processed sensor data from the WEAD comprising data sensed by the at least one sensor when the WEAD is worn by a human user and processed by the SLM to obtain the processed sensor data; processing the received processed sensor data; and providing a suggested amount of mandibular advancement or retraction for the human user based on the result of the processing, the suggested amount including an incremental adjustment to be made to the adjustment mechanism to adjust a position of the second portion relative to the first portion when the WEAD is worn by the human user.

Clause 12: The method of Clause 11, further comprising providing the WEAD.

Clause 13: The method of Clause 12, wherein providing the WEAD comprises creating the first portion, the second portion, and a housing to house the at least one sensor and the processor comprising the SLM from an intraoral scan of the human user.

Clause 14: The method of any of Clauses 11-13, wherein processing the received processed sensor data is done in the cloud.

Clause 15: The method of any of Clauses 11-14, wherein the processed sensor data from the WEAD is received by a computing device.

Clause 16: A wearable device comprising: a first portion; and a housing comprising at least one sensor, a power source, wireless communications circuitry, and a processor comprising a small language model (SLM), the housing coupled to the first portion such that the at least one sensor is positioned relative to a roof of an oral cavity when the first portion and the housing are custom-fit to and worn on the upper teeth of the human user, wherein, in use when the wearable device is worn by the human user, the at least one sensor is configured to sense at least one of a linear acceleration or a rotational acceleration and provide data for processing by the SLM to detect a concussive event.

Clause 17: The wearable device of Clause 16, wherein the first portion is custom-fit to the human user in a boil-and-bite process.

Clause 18: The wearable device of Clause 16 or Clause 17, wherein the at least one sensor comprises at least one of the group consisting of: a photoplethysmography (PPG) sensor, an accelerometer, a gyroscope, a microphone, a temperature sensor, and a piezo sensor.

Clause 19: A system comprising: the wearable device of any of Clauses 16-18; and an application operating on a computing device, the computing device configured to receive SLM-processed sensor data from the wearable device via the wireless communications circuitry.

Clause 20: The system of Clause 19, further comprising a cloud-based computing system configured to receive the SLM-processed sensor data from the application operating on the computing device.

It should be appreciated that dimensional relationships among individual elements in the attached drawings are illustrated only for ease of understanding, but not to limit the actual scale. Furthermore, particular dimensions, ranges, values, and example numbers given herein are examples only and can vary by 1 percent, 5 percent, 10 percent, 20 percent, 25 percent, 30 percent, and up to 50 percent in various embodiments without departing from the scope of the disclosure.

Use of the term “or” in this disclosure is inclusive, e.g., “and/or,” unless otherwise explicitly stated here. For example, “A can comprise B or C” can mean that A comprises B, A, comprises C, and A comprises B and C. Use of the term “step” or “operation” does not imply or require any particular order of any disclosed activities, such that the activities of any step can be reordered, include additional intervening activities, or omit one or more activities, unless otherwise explicitly stated here.

Some embodiments can include, interface with, or otherwise utilize technologies that are rapidly developing and evolving, and the intentional of the inventors is to capture future developments and evolutions of these technologies, even if not explicitly disclosed herein, at least to the extend such future developments and evolutions of these technologies are consistent with this disclosure or constructively support embodiments disclosed and discussed herein.

While embodiments of this disclosure are amenable to various modifications and alternative forms, specifics thereof have been shown by way of example in the drawings and will be described in detail. It should be understood, however, that the intention is not to limit the invention to the particular example embodiments described. On the contrary, the intention is to cover all modifications, equivalents, and alternatives falling within the spirit and scope of the disclosure as defined by the appended claims.

1. A wearable electronic airway device (WEAD) comprising:
a first portion comprising:
a housing comprising at least one sensor, a power source, wireless communications circuitry, and a processor comprising a small language model (SLM), and
a first component of an adjustment mechanism,
a second portion comprising a second component of the adjustment mechanism, the second component configured to selectively engage with the first component to adjustably couple the first portion and the second portion,
wherein, in use by a human user having upper teeth and lower teeth, the first portion is configured to be worn on the upper teeth such that the at least one sensor is positioned adjacent to a tooth line on a roof of an oral cavity, the lower portion is configured to be worn on the lower teeth, and mandibular advancement or retraction is provided by adjusting a position of the second portion relative to the first portion using the adjustment mechanism.

2. The WEAD of claim 1, wherein the housing is removable from the first portion and the WEAD.

3. The WEAD of claim 1, wherein the at least one sensor comprises at least one of the group consisting of: a photoplethysmography (PPG) sensor, an accelerometer, a gyroscope, a microphone, a temperature sensor, and a piezo sensor.

4. The WEAD of claim 1, wherein the power source is a rechargeable battery.

5. The WEAD of claim 1, wherein one of the first component or the second component of the adjustment mechanism comprises a titration screw, and the other of the first component or the second component comprises a ratchet.

6. The WEAD of claim 5, wherein the titration screw is incrementally adjustable relative to the ratchet, and wherein a recommended amount of incremental adjustment for the human user is provided at least in part by the SLM based on sensor data from the at least one sensor.

7. A system comprising:
the WEAD of claim 1; and
an application operating on a computing device, the computing device configured to receive SLM-processed sensor data from the WEAD via the wireless communications circuitry.

8. The system of claim 7, further comprising a cloud-based computing system configured to receive the SLM-processed sensor data from the application operating on the computing device.

9. The system of claim 8, wherein at least one of the computing device or the cloud-based computing system is configured to further process the SLM-processed sensor data to obtain further processed sensor data.

10. The system of claim 9, wherein at least one of the computing device or the cloud-based computing system is configured to provide a recommended adjustment of the adjustment mechanism to change the position of the second portion relative to the first portion based on at least one of the SLM-processed sensor data or the further processed sensor data.

11. A method of managing a wearable electronic airway device (WEAD), the WEAD comprising a first portion and a second portion, the first portion including at least one sensor, a processor comprising a small language model (SLM), and a first component of an adjustment mechanism and the second portion comprising a second component of the adjustment mechanism, the second component configured to selectively engage with the first component to adjustably couple the first portion and the second portion, the method comprising:
receiving processed sensor data from the WEAD comprising data sensed by the at least one sensor when the WEAD is worn by a human user and processed by the SLM to obtain the processed sensor data;
processing the received processed sensor data; and
providing a suggested amount of mandibular advancement or retraction for the human user based on the result of the processing, the suggested amount including an incremental adjustment to be made to the adjustment mechanism to adjust a position of the second portion relative to the first portion when the WEAD is worn by the human user.

12. The method of claim 11, further comprising providing the WEAD.

13. The method of claim 12, wherein providing the WEAD comprises creating the first portion, the second portion, and a housing to house the at least one sensor and the processor comprising the SLM from an intraoral scan of the human user.

14. The method of claim 11, wherein processing the received processed sensor data is done in the cloud.

15. The method of claim 11, wherein the processed sensor data from the WEAD is received by a computing device.

16. A wearable device comprising:
a first portion; and
a housing comprising at least one sensor, a power source, wireless communications circuitry, and a processor comprising a small language model (SLM), the housing coupled to the first portion such that the at least one sensor is positioned relative to a roof of an oral cavity when the first portion and the housing are custom-fit to and worn on the upper teeth of the human user,
wherein, in use when the wearable device is worn by the human user, the at least one sensor is configured to sense at least one of a linear acceleration or a rotational acceleration and provide data for processing by the SLM to detect a concussive event.

17. The wearable device of claim 16, wherein the first portion is custom-fit to the human user in a boil-and-bite process.

18. The wearable device of claim 16, wherein the at least one sensor comprises at least one of the group consisting of: a photoplethysmography (PPG) sensor, an accelerometer, a gyroscope, a microphone, a temperature sensor, and a piezo sensor.

19. A system comprising:
the wearable device of claim 16; and
an application operating on a computing device, the computing device configured to receive SLM-processed sensor data from the wearable device via the wireless communications circuitry.

20. The system of claim 19, further comprising a cloud-based computing system configured to receive the SLM-processed sensor data from the application operating on the computing device.
